(19) United States
(12) Patent Application Publication
(10) Pub. No.: US 2025/0065102 A1
(43) Pub. Date: Feb. 27, 2025

(54) METHODS AND APPARATUS FOR PREDICTING DECOUPLING DURING NON-CARDIAC MEDICAL PROCEDURES

(71) Applicant: Abiomed, Inc., Danvers, MA (US)

(72) Inventors: Jin Kwang Kim, Danvers, MA (US); Daniel Podlisny, Danvers, MA (US); Sean Clifford, Danvers, MA (US)

(21) Appl. No.: 18/812,211

(22) Filed: Aug. 22, 2024

Related U.S. Application Data
(60) Provisional application No. 63/578,099, filed on Aug. 22, 2023.

(51) Int. Cl. A61M 60/174; A61M 60/216; A61M 60/531; A61M 60/585; G16H 40/63; G16H 50/70

(52) U.S. Cl. CPC A61M 60/174; A61M 60/216; A61M 60/531; A61M 60/585; G16H 40/63; G16H 50/70

(57) ABSTRACT

Methods and apparatus for predicting decoupling during a non-cardiac medical procedure are provided. The method includes receiving one or more patient characteristics associated with a patient scheduled for a non-cardiac medical procedure, providing the one or more patient characteristics as input to a machine learning model trained to output a decoupling prediction, processing, using at least one computer processor, the one or more patient characteristics using the machine learning model to output a decoupling prediction for the patient, wherein the decoupling prediction is associated with the non-cardiac medical procedure, and displaying, on a user interface, an indication of the decoupling prediction for the patient output from the machine learning model.

### RELATED APPLICATIONS

This application claims the benefit under 35 U.S.C. § 119 (e) to U.S. Provisional Patent Application No. 63/578,099, filed Aug. 22, 2023, and titled, “METHODS AND APPARATUS FOR PREDICTING DECOUPLING DURING NON-CARDIAC MEDICAL PROCEDURES,” the entire contents of which is incorporated by reference herein.

### FIELD OF THE INVENTION

This disclosure relates to techniques for predicting cardiac decoupling during a non-cardiac medical procedure.

### BACKGROUND

Cardiovascular diseases are a leading cause of morbidity, mortality, and burden on global healthcare. A variety of treatment modalities have been developed for heart health, ranging from pharmaceuticals to mechanical devices and transplantation. Temporary cardiac support devices, such as heart pump systems (also referred to as “intracardiac blood pumps”), provide hemodynamic support and facilitate heart recovery. Intracardiac blood pumps have traditionally been used to temporarily assist the pumping function of a patient's heart during emergent cardiac procedures, such as a stent placement, performed after the patient suffers a heart attack, cardiac arrest, and/or cardiogenic shock. Intracardiac blood pumps also may be used to take the load off of a patient's heart to allow the heart to recover from such a cardiac procedure or from a heart attack, cardiac arrest, cardiogenic shock, or heart damage (e.g., caused by a viral infection). In that regard, an intracardiac blood pump can be introduced into the heart either surgically or percutaneously and used to deliver blood from one location in the heart or circulatory system to another location in the heart or circulatory system. For example, when deployed in the left heart, an intracardiac blood pump can pump blood from the left ventricle of the heart into the aorta. Likewise, when deployed in the right heart, an intracardiac blood pump can pump blood from the inferior vena cava into the pulmonary artery. Intracardiac pumps can be powered by a motor located outside of the patient's body via an elongate drive shaft (or drive cable) or by an onboard motor located inside the patient's body. Examples of such devices include the Impella™ family of devices (Abiomed, Inc., Danvers, MA).

### SUMMARY

Intracardiac blood pumps may be used during medical procedures (e.g., non-cardiac surgery (NCS)) to provide cardiac support to patients who are otherwise high risk (e.g., due to their age, medical history, co-morbidities, etc.). For example, in some instances, a patient requiring a medical procedure who may have been turned down for the procedure based on a risk that the patient may experience an adverse outcome (e.g., the procedure itself may cause the patient to experience hemodynamic instability during and/or following the procedure and/or may lead to the patient's death), may become eligible for such a procedure if the risk is reduced by using an intracardiac blood pump before, during or after the procedure.

When using an intracardiac blood pump, decoupling occurs when the patient's heart is relying on the intracardiac blood pump for cardiac support instead of their native heart function. Some embodiments of the present disclosure relate to techniques for analyzing pressure signals within a patient's heart to detect decoupling during intracardiac blood pump use. Some embodiments of the present disclosure relate to using patient characteristics (e.g., age, sex, cardiac function, etc.) for patients who experienced decoupling during non-cardiac surgery to inform and/or train a predictive algorithm or model about the likelihood that other patients will experience decoupling during a non-cardiac surgery. Such a predictive algorithm or model may be used, among other things, as a clinical decision support tool to provide a recommendation to a healthcare provider about the efficacy of using an intracardiac blood pump during a non-cardiac medical procedure (e.g., non-cardiac surgery). In some embodiments, the output of the predictive algorithm/model may be used as a predictor of the patient's chance of survival without support by an intracardiac blood pump.

In some embodiments, a computer-implemented method is provided. The computer-implemented method includes receiving one or more patient characteristics associated with a patient scheduled for a non-cardiac medical procedure, providing the one or more patient characteristics as input to a machine learning model trained to output a decoupling prediction, processing, using at least one computer processor, the one or more patient characteristics using the machine learning model to output a decoupling prediction for the patient, wherein the decoupling prediction is associated with the non-cardiac medical procedure, and displaying, on a user interface, an indication of the decoupling prediction for the patient output from the machine learning model.

In one aspect, the one or more patient characteristics include one or more of physical characteristics, medical history information, medication information, or physiological metrics associated with the patient. In another aspect, receiving one or more patient characteristics comprises receiving the one or more patient characteristics from an electronic medical record associated with the patient. In another aspect, the machine learning model is trained using training data from a plurality of patients having undergone different types of non-cardiac medical procedures. In another aspect, displaying an indication of the decoupling prediction for the patient comprises displaying a predicted amount of decoupling that the patient is likely to experience during the non-cardiac medical procedure.

In another aspect, the non-cardiac medical procedure has an expected procedure duration, and wherein displaying a predicted amount of decoupling comprises displaying the predicted amount of decoupling as a percentage of the expected procedure duration. In another aspect, the computer-implemented method further includes determining, when the predicted amount of decoupling exceeds a threshold value, that the patient would benefit from use of an intracardiac blood pump in association with the non-cardiac medical procedure, and displaying, on the user interface, a recommendation associated with the use of the intracardiac blood pump. In another aspect, the recommendation includes a time period during which use of the intracardiac blood pump would be beneficial to the patient. In another aspect, the time period includes one or more of before the non-cardiac medical procedure, during the non-cardiac medical procedure, or after the non-cardiac medical procedure.

In some embodiments, a controller for a heart pump system is provided. The controller includes at least one hardware processor configured to receive one or more patient characteristics associated with a patient scheduled for a non-cardiac medical procedure, provide the one or more patient characteristics as input to a machine learning model trained to output a decoupling prediction for the patient, wherein the decoupling prediction is associated with the non-cardiac medical procedure, and display, on a user interface associated with the heart pump system, an indication of the decoupling prediction for the patient output from the machine learning model.

In some embodiments, a method of treating a patient is provided. The method includes receiving a decoupling prediction for a patient scheduled for a non-cardiac medical procedure, wherein the decoupling prediction is output from a trained machine learning model in response to providing one or more patient characteristics associated with the patient as input, determining, based on the decoupling prediction, a period of time when the patient would benefit from use of an intracardiac blood pump associated with the non-cardiac medical procedure, inserting the intracardiac blood pump into a heart of the patient during the determined period of time, and performing the non-cardiac medical procedure to treat the patient.

In one aspect, the one or more patient characteristics include one or more of physical characteristics, medical history information, medication information, or physiological metrics associated with the patient. In another aspect, the one or more patient characteristics are provided as input to the trained machine learning model from an electronic medical record associated with the patient. In another aspect, the trained machine learning model is trained using training data from a plurality of patients having undergone different types of non-cardiac medical procedures. In another aspect, the period of time includes one or more of before the non-cardiac medical procedure, during the non-cardiac medical procedure, or after the non-cardiac medical procedure. In another aspect, the method further includes tracking during the non-cardiac medical procedure an amount of decoupling experienced by the patient, and retraining the trained machine learning model based, at least in part, on the amount of decoupling experienced by the patient and the one or more patient characteristics.

In some embodiments, a computer-implemented method for training a machine learning model to predict decoupling during a non-cardiac medical procedure is provided. The computer-implemented method includes receiving patient data for a plurality of patients, wherein each of the plurality of patients underwent a non-cardiac medical procedure associated an intracardiac blood pump, analyzing, by at least one hardware processor, the patient data to detect one or more decoupling events during the non-cardiac medical procedure associated with each patient of the plurality of patients, training the machine learning model based, at least in part, on the one or more decoupling events detected during a non-cardiac medical procedure and one or more patient characteristics associated with the patient associated with the non-cardiac medical procedure, and outputting the trained machine learning model for predicting decoupling in one or more patients not associated with the patient data.

In one aspect, the patient data includes the one or more patient characteristics associated with a patient and pressure information sensed by and/or derived from one or more pressure sensors of a mechanical circulatory support device that includes the intracardiac blood pump. In another aspect, the pressure information includes a left ventricular pressure signal and an aortic pressure signal. In another aspect, analyzing the patient data to detect one or more decoupling events comprises detecting a decoupling event when a peak of the aortic pressure signal is greater than a peak of the left ventricular pressure signal within a cardiac cycle. In another aspect, the method further includes determining an amount of decoupling for the patient during the non-cardiac medical procedure based on the detected one or more decoupling events, and training the machine learning model based, at least in part, on the one or more decoupling events comprises training the machine learning model based, at least in part, on the amount of decoupling for the patient during the non-cardiac medical procedure. In another aspect, receiving patient data for a plurality of patients comprises receiving the patient data from corresponding electronic medical records associated with the plurality of patients.

### BRIEF DESCRIPTION OF DRAWINGS

FIG. 1A illustrates a pump system in accordance with some embodiments of the present disclosure.

FIG. 1B is a cross-sectional view of a portion of the pump system of FIG. 1A.

FIG. 2 illustrates a pump system in accordance with some embodiments of the present disclosure.

FIG. 3 illustrates a pump system in accordance with some embodiments of the present disclosure.

FIG. 4 is a flowchart of a process for updating an algorithm or model used to predict decoupling during a non-cardiac procedure, in accordance with some embodiments of the present disclosure.

FIG. 5 illustrates example pressure signals that may be used to detect decoupling during a medical procedure, in accordance with some embodiments of the present disclosure.

FIG. 6 illustrates a process for detecting decoupling during a medical procedure using comparisons of pressure signals, in accordance with some embodiments of the present disclosure.

FIG. 7 is a flowchart of a process for using an updated decoupling algorithm/model to predict an amount of decoupling during a non-cardiac medical procedure, in accordance with some embodiments of the present disclosure.

FIG. 8 is a flowchart of a process for using an intracardiac blood pump to reduce cardiac risk during performance of a non-cardiac medical procedure.

### DETAILED DESCRIPTION

Use of an intracardiac blood pump to provide cardiac support to a patient during non-cardiac medical procedures (e.g., non-cardiac surgery such as cyst removal, knee replacement, bariatric surgery, etc.) may reduce the risk that the patient will experience a cardiac-related adverse event during the procedure. By reducing such risk, the patient may become eligible for procedures that without the use of the blood pump may be seen as too risky by healthcare providers. Determining whether the cardiac benefits of using an intracardiac blood pump during a non-cardiac medical procedure would sufficiently outweigh the risks involved with placing the device in the patient's heart before, during, and/or after the procedure may depend on several factors. Decoupling is a state during which the heart's native function of ejecting blood through the heart valves is diminished substantially such that circulation of blood through a patient's heart is maintained primarily or entirely based on the continuous flow of blood pumped through the intracardiac blood pump placed in the patient's heart. Some embodiments of the present disclosure relate to predicting whether and/or the extent to which decoupling is likely to occur during a non-cardiac medical procedure for a particular patient based, at least in part, on one or more patient characteristics. By estimating the likelihood and/or extent of decoupling during a possible non-cardiac surgery, a physician or other healthcare provider may be better informed when deciding whether use of an intracardiac blood pump before, during and/or after the procedure would be beneficial.

To provide an overall understanding of the systems, methods, and devices described herein, certain illustrative examples will be described. Although various examples may describe specific medical procedures and/or uses of intracardiac blood pumps, it will be understood that the technology described herein may be employed in any suitable context.

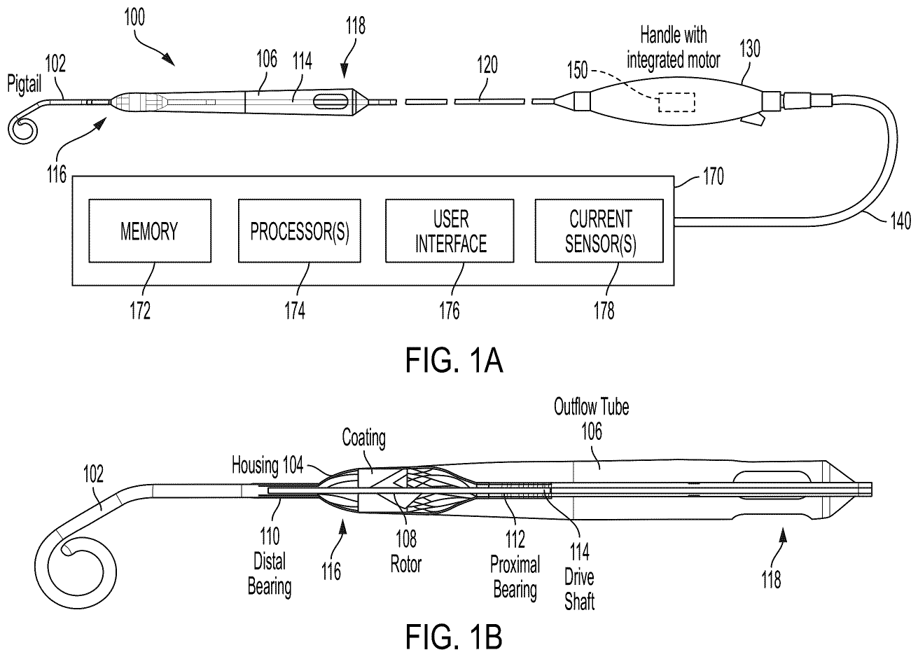

A pump system including pump 100 for use with some embodiments of the present disclosure is shown in FIGS. 1A and 1B. As shown, the pump 100 may be coupled to a control unit 170. Pump 100 may include a distal atraumatic tip 102, a pump housing 104 surrounding a rotor 108, an outflow tube 106, distal bearing 110, proximal bearing 112, inlet 116, outlet 118, catheter 120, handle 130, cable 140, and motor 150. As will be appreciated, although shown with an atraumatic tip, in some embodiments, the pump may not include such a tip. Pump housing 104 may be configured as a frame structure formed by a mesh with openings which may, at least in part, be covered by an elastic material. As will be appreciated, although shown as a frame structure, in some embodiments, the pump housing may be solid. A proximal portion of pump housing 104 may extend into and be mounted in the hollow interior of outflow tube 106, and a distal portion of pump housing 104 may extend distally beyond the distal end of outflow tube 106. The exposed openings in the pump housing 104 extending distally beyond outflow tube 106 may form the inlet 116 of pump 100. The proximal end of outflow tube 106 may include a plurality of openings that form the outlet 118 of pump 100. Rotor 108 may be rotationally mounted between distal bearing 110 and proximal bearing 112, and may be coupled to a distal end of drive shaft 114. Drive shaft 114 may be flexible and may extend through catheter 120, through the hollow interior of outflow tube 106, into handle 130 and is coupled to motor 150, which is housed in handle 130. The proximal end of handle 130 may be coupled via cable 140 to control unit 170. A fluid may be circulated through the catheter 120 proximate to the drive shaft 114 and in the space surrounding the distal bearing 110 and proximal bearing 112 to lubricate those components and reduce friction during operation of the pump 100.

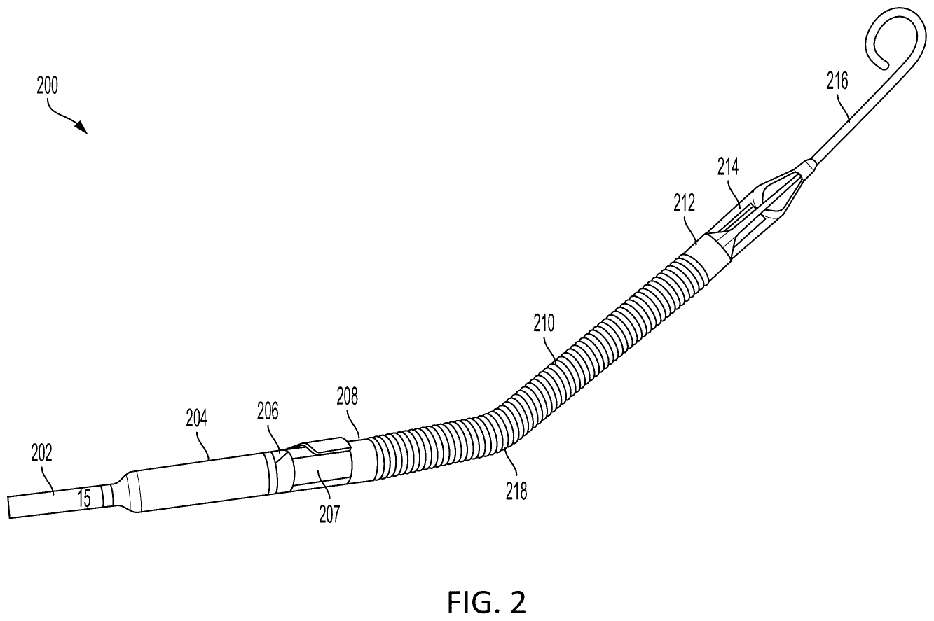

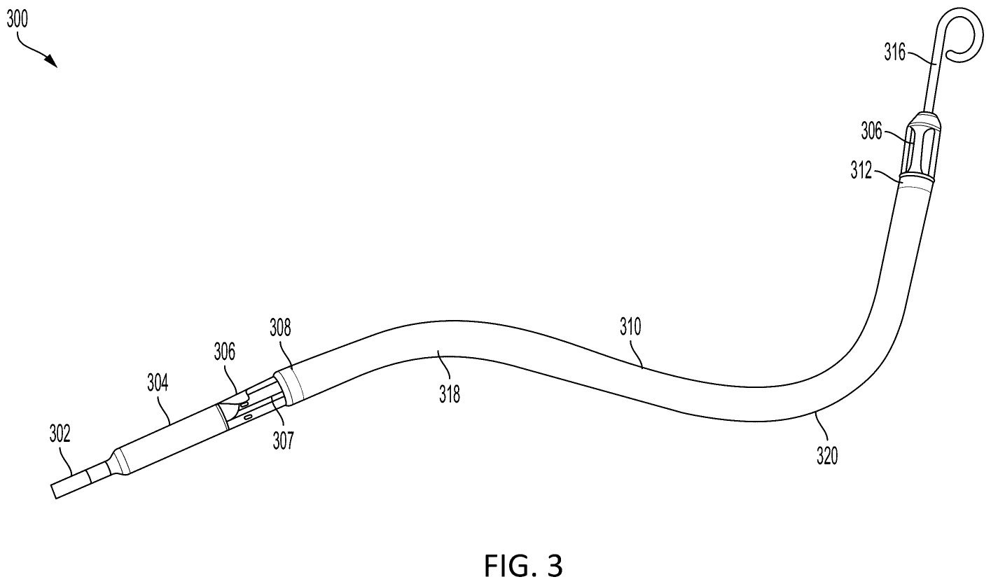

Control unit 170 may include one or more memory 172, one or more processors 174, a user interface 176, and one or more sensors, such as current sensors 178. Processor(s) 174 may comprise one or more microcontrollers, one or more microprocessors, one or more application specific integrated circuits (ASICs), one or more digital signal processors, program memory, or other computing components. Processor(s) 174 may be communicatively coupled to the other components (e.g., memory 172, user interface 176, current sensor(s) 178) of control unit 170 and may be configured to control one or more operations of pump 100. As will be appreciated, although control unit 170 is shown connected to pump 100, control unit may be connected to pump 200 or pump 300 described in connection with FIGS. 2 and 3. As a non-limiting example, control unit 170 may be implemented as an Automated Impella Controller™ from ABIOMED, Inc., Danvers, MA. In some aspects, memory 172 is included as a portion of processor(s) 174 rather than being provided as a separate component.

During operation, processor(s) 174 may be configured to control the electrical power delivered to motor 150 (e.g., by controlling a power supply (not shown)) by a power supply line (not shown) in cable 140, thereby controlling the speed of the motor 150. Current sensor(s) 178 may be configured to sense motor current associated with an operating state of the motor 150, and processor(s) 174 may be configured to receive the output of current sensor(s) 408 as a motor current signal. Processor(s) 174 may further be configured to determine a flow through the pump 100 based, at least in part, on the motor current signal and the motor speed, as described in more detail herein. Current sensor(s) 178 may be included in control unit 170 or may be located along any portion of the power supply line in cable 140. Additionally or alternatively, current sensor(s) 178 may be included in motor 150 and processor(s) 174 may be configured to receive the motor current signal via a data line (not shown) in cable 140 coupled to processor(s) 174 and motor 150.

Memory 172 may be configured to store computer-readable instructions and other information for various functions of the components of control unit 170. In one aspect, memory 172 includes volatile and/or non-volatile memory, such as, an electrically erasable programmable read-only memory (EEPROM).

User interface 176 may be configured to receive user input via one or more buttons, switches, knobs, etc. Additionally, user interface 176 may include a display configured to display information and one or more indicators, such as light indicators, audio indicators, etc., for conveying information and/or providing alerts regarding the operation of pump 100.

Pump 100 may be designed to be insertable into a patient's body, e.g., into a left ventricle of the heart, such as via an introducer system. Although some of the systems and/or methods disclosed herein are described for modulating a pump speed of a pump inserted into the left ventricle of a heart, it should be appreciated that the systems and/or methods described herein may also be applied to other types of ventricular support systems, such as a ventricular support system inserted into the right ventricle of the heart. In one aspect, housing 104, rotor 108, and outflow tube 106 may be radially compressible to enable pump 100 to achieve a relatively small outer diameter of, for example, 9 Fr (3 mm) during insertion. When pump 100 is inserted into the patient, e.g., into a left ventricle, handle 130 and motor 150 may remain disposed outside the patient. As will be appreciated, in other embodiments, the motor of the pump system may be disposed inside the patient upon insertion. During operation, motor 150 is controlled by processor(s) 404 to drive rotation of drive shaft 114 and rotor 108 to convey blood from inlet 116 to outlet 118. It is to be appreciated that rotor 108 may be rotated by motor 150 in reverse to convey blood in the opposite direction (in this case, the openings of 118 form the inlet and the openings of 116 form the outlet). In one aspect, pump 100 may be configured to be used for weeks to months to years to support the heart function of a patient with chronic heart failure, though it should be understood that the technology described herein is not limited to any particular types of procedures and/or use durations.

A blood pump system for use with some embodiments is shown in FIG. 2. FIG. 2 depicts an exemplary intracardiac blood pump assembly 200 adapted for left heart support, in accordance with aspects of the disclosure. As shown in FIG. 2, an intracardiac blood pump assembly adapted for left heart support may include an elongate catheter 202, a motor 204, a cannula 210, a blood inflow cage 214 arranged at or near the distal end 212 of the cannula 210, a blood outflow cage 206 arranged at or near the proximal end 208 of the cannula 210, and an optional atraumatic extension 216 arranged at the distal end of the blood inflow cage 214.

In some aspects of the technology, motor 204 may be configured to rotatably drive an impeller (not shown), thereby generating suction sufficient to draw blood into cannula 210 through the blood inflow cage 214, and to expel the blood out of cannula 210 through the blood outflow cage 206. In that regard, the impeller may be positioned distal of the blood outflow cage 206, for example, within the proximal end 208 of the cannula 210 or within a pump housing 207 coupled to the proximal end 208 of the cannula 210. In some aspects of the technology, rather than the impeller being driven by an onboard motor 204, the impeller may instead be coupled to an elongate drive shaft (or drive cable) which is driven by a motor located external to the patient.

Catheter 202 may house electrical lines coupling the motor 204 to one or more electrical controllers and/or sensors. Alternatively, where the impeller is driven by an external motor, an elongate drive shaft may pass through catheter 202. Catheter 202 may also include a purge fluid conduit, a lumen configured to receive a guidewire, etc.

The blood inflow cage 214 may include one or more apertures or openings configured to allow blood to be drawn into cannula 210 when the motor 204 is operating. Likewise, blood outflow cage 206 may include one or more apertures or openings configured to allow blood to flow from the cannula 210 out of the intracardiac blood pump assembly 200. Blood inflow cage 214 and outflow cage 206 may be composed of any suitable bio-compatible material(s). For example, blood inflow cage 214 and/or blood outflow cage 206 may be formed out of bio-compatible metals such as stainless steel, titanium, or biocompatible polymers such as polyurethane. In addition, the surfaces of blood inflow cage 214 and/or blood outflow cage 206 may be treated in various ways, including, but not limited to etching, texturing, or coating or plating with another material. For example, the surfaces of blood inflow cage 214 and/or blood outflow cage 206 may be laser textured.

Cannula 210 may include a flexible hose portion. For example, cannula 210 may be composed, at least in part, of a polyurethane material. In addition, cannula 210 may include a shape-memory material. For example, cannula 210 may comprise a combination of a polyurethane material and one or more strands or coils of a shape-memory material such as Nitinol. Cannula 210 may be formed such that it includes one or more bends or curves in its relaxed state, or it may be configured to be straight in its relaxed state. In that regard, as shown in the exemplary arrangement of FIG. 2, the cannula 210 may have a single pre-formed anatomical bend 218 based on the portion of the left heart in which it is intended to operate. Despite this bend 218, the cannula 210 may nevertheless also be flexible, and may thus be capable of straightening (e.g., during insertion over a guidewire), or bending further (e.g., in a patient whose anatomy has tighter dimensions). Further in that regard, cannula 210 may include a shape-memory material configured to allow the cannula 210 to be a different shape (e.g., straight or mostly straight) at room temperatures, and to form bend 218 once the shape-memory material is exposed to the heat of a patient's body.

Atraumatic extension 216 may assist with stabilizing and positioning the intracardiac blood pump assembly 200 in the correct position in the patient's heart. Atraumatic extension 216 may be solid or tubular. If tubular, atraumatic extension 216 may be configured to allow a guidewire to be passed through it to further assist in the positioning of the intracardiac blood pump assembly 200. Atraumatic extension 216 may be any suitable size. For example, atraumatic extension 216 may have an outer diameter in the range of 4-8 Fr. Atraumatic extension 216 may be composed, at least in part, of a flexible material, and may be any suitable shape or configuration such as a straight configuration, a partially curved configuration, a pigtail-shaped configuration as shown in the example of FIG. 2, etc. Atraumatic extension 216 may also have sections with different stiffnesses. For example, atraumatic extension 216 may include a proximal section that is stiff enough to prevent it from buckling, thereby keeping the blood inflow cage 214 in the desired location, and a distal section that is softer and has a lower stiffness, thereby providing an atraumatic tip for contact with a wall of the patient's heart and to allow for guidewire loading. In such a case, the proximal and distal sections of the atraumatic extension 216 may be composed of different materials, or may be composed of the same material with the proximal and distal sections being treated to provide different stiffnesses.

Notwithstanding the foregoing, as mentioned above, atraumatic extension 216 is an optional structure. In that regard, the present technology may also be used with intracardiac blood pump assemblies and other intracardiac devices that include extensions of different types, shapes, materials, and qualities. Likewise, the present technology may be used with intracardiac blood pump assemblies and other intracardiac devices that have no distal extensions of any kind.

As described herein, the intracardiac blood pump assembly 200 may be inserted percutaneously. For example, when used for left heart support, intracardiac blood pump assembly 200 may be inserted via a catheterization procedure through the femoral artery or axillary artery, into the aorta, across the aortic valve, and into the left ventricle. Once positioned in this way, the intracardiac blood pump assembly 200 may deliver blood from the blood inflow cage 214, which sits inside the left ventricle, through cannula 210, to the blood outflow cage 206, which sits inside the ascending aorta. In some aspects of the technology, intracardiac blood pump assembly 200 may be configured such that bend 218 will rest against a predetermined portion of the patient's heart when the intracardiac blood pump assembly 200 is in a desired location. Likewise, the atraumatic extension 216 may be configured such that it rests against a different predetermined portion of the patient's heart when the intracardiac blood pump assembly 200 is in the desired location.

FIG. 3 depicts an exemplary intracardiac blood pump assembly 300 adapted for right heart support, in accordance with aspects of the disclosure. As shown in FIG. 3, an intracardiac blood pump assembly adapted for right heart support may include an elongate catheter 302, a motor 304, a cannula 310, a blood inflow cage 314 arranged at or near the proximal end 308 of the cannula 310, a blood outflow cage 306 arranged at or near the distal end 312 of the cannula 310, and an optional atraumatic extension 316 arranged at the distal end of the blood outflow cage 306.

As with the exemplary assembly of FIG. 2, motor 304 may be configured to rotatably drive an impeller (not shown), thereby generating suction sufficient to draw blood into cannula 310 through the blood inflow cage 314, and to expel the blood out of cannula 310 through the blood outflow cage 306. In that regard, the impeller may be positioned distal of the blood inflow cage 314, for example, within the proximal end 308 of the cannula 310 or within a pump housing 307 coupled to the proximal end 308 of the cannula 310. Here as well, in some aspects of the technology, rather than the impeller being driven by an onboard motor 304, the impeller may instead be coupled to an elongate drive shaft (or drive cable) which is driven by a motor located external to the patient.

The cannula 310 of FIG. 3 may serve the same purpose, and may have the same properties and features described above with respect to cannula 210 of FIG. 2. However, as shown in the exemplary arrangement of FIG. 3, the cannula 310 may have two pre-formed anatomical bends 318 and 320 based on the portion of the right heart in which it is intended to operate. Here again, despite the existence of bends 318 and 320, the cannula 310 may nevertheless also be flexible, and may thus be capable of straightening (e.g., during insertion over a guidewire), or bending further (e.g., in a patient whose anatomy has tighter dimensions). Further in that regard, cannula 310 may include a shape-memory material configured to allow the cannula 310 to be a different shape (e.g., straight or mostly straight) at room temperatures, and to form bends 318 and/or 320 once the shape-memory material is exposed to the heat of a patient's body.

The catheter 302 and atraumatic extension 316 of FIG. 3 may serve the same purpose and may have the same properties and features described above with respect to catheter 202 and atraumatic extension 216 of FIG. 2. Likewise, other than being located at opposite ends of the cannula from those of FIG. 2, the blood inflow cage 314 and blood outflow cage 306 of FIG. 3 may be similar to the blood inflow cage 214 and blood outflow cage 206 of FIG. 2, and thus may have the same properties and features described above.

Like the exemplary assembly of FIG. 2, the intracardiac blood pump assembly 300 of FIG. 3 may also be inserted percutaneously. For example, when used for right heart support, intracardiac blood pump assembly 300 may be inserted via a catheterization procedure through the femoral vein, into the inferior vena cava, through the right atrium, across the tricuspid valve, into the right ventricle, through the pulmonary valve, and into the pulmonary artery. Once positioned in this way, the intracardiac blood pump assembly 300 may deliver blood from the blood inflow cage 314, which sits inside the inferior vena cava, through cannula 310, to the blood outflow cage 306, which sits inside the pulmonary artery.

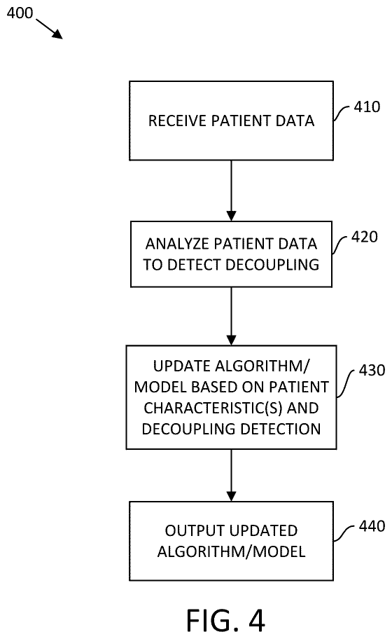

FIG. 4 illustrates a flowchart of a process 400 for generating and/or updating an algorithm or model to output a decoupling prediction, in accordance with some embodiments of the present disclosure. Process 400 may begin in act 410, where patient data is received. The received patient data may be associated with patients that had a medical procedure (e.g., a non-cardiac surgery) during which an intracardiac blood pump was used. The patient data may be received in any suitable way. For instance, in some embodiments, the patient data may be received from one or more electronic patient medical records (e.g., electronic health records (EHRs). The patient data may include one or more patient characteristics and pressure information sensed by and/or derived from one or more pressure sensors of an MCS device that includes the intracardiac blood pump. The pressure information may include left ventricular pressure (LVP) information and aortic pressure (AOP) information, which may be used to detect decoupling during the medical procedure, as discussed in further detail herein.

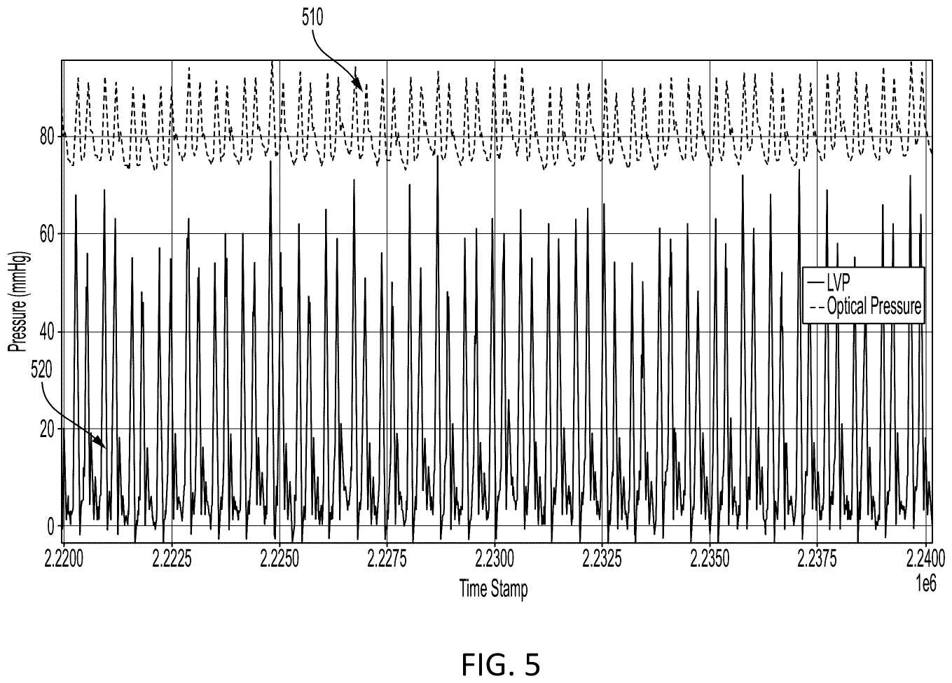

Process 400 may then proceed to act 420, where the patient data may be analyzed to detect decoupling during the non-cardiac procedure. FIG. 5 illustrates an example of pressure information that may be included as patient data in act 410 and analyzed in act 420 to detect decoupling. As shown in FIG. 5, pressure information includes AOP information 510 and LVP information 520. Each of AOP information 510 and LVP information 520 is shown as a pressure waveform sensed by one or more pressure sensors over time. In the example shown in FIG. 5, the AOP information 510 was sensed by an optical pressure sensor arranged on the intracardiac blood pump and the LVP information 520 was derived based on the AOP information 510 and other information (e.g., motor current and/or motor speed of the intracardiac blood pump). It should be appreciated however, that the pressure information may be sensed using more than one sensor and embodiments of the present disclosure are not limited in this respect.

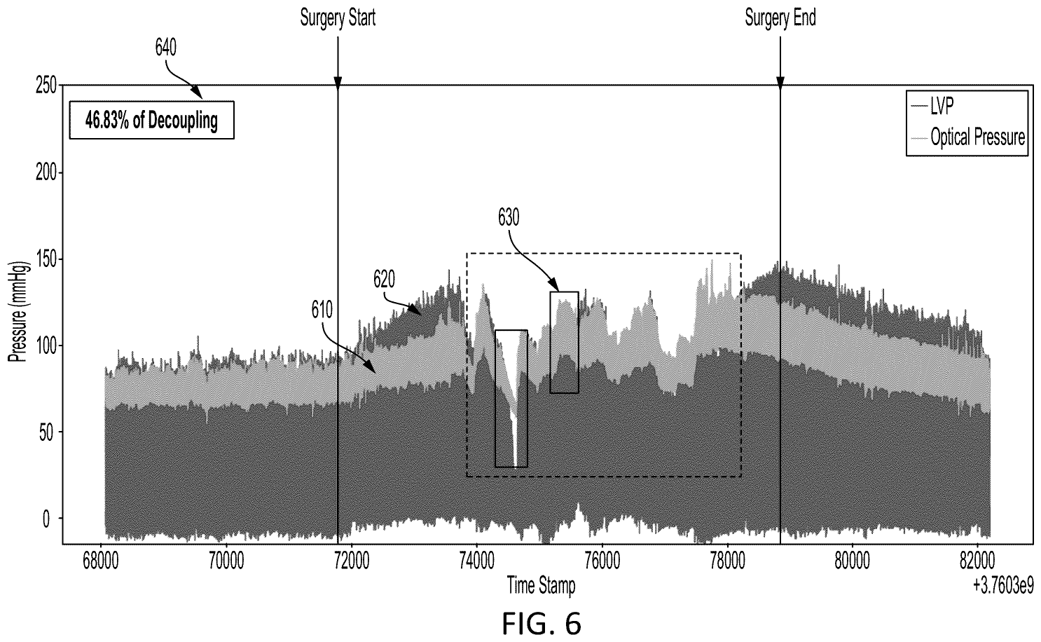

FIG. 6 schematically shows an example technique for detecting decoupling during a non-cardiac medical procedure in accordance with some embodiments of the present disclosure. In the example shown in FIG. 6, a peak-to-peak comparison of AOP signal 610 and LVP signal 620 in one cardiac cycle is performed and decoupling is detected when the peak AOP signal is greater than the peak LVP signal within the cardiac cycle. Example portions of the waveform in which the peak AOP signal is greater than the peak LVP signal is shown in FIG. 6 using bounding boxes 630. FIG. 6 also shows the start time and end time for a non-cardiac medical procedure (e.g., non-cardiac surgery), collectively forming a procedure window. In some embodiments, an amount (e.g., a percentage) of decoupling detected within the procedure window is shown as indicator 640. As described herein, a higher percentage of decoupling within the procedure window may represent a patient's increased reliance on the intracardiac pump during the procedure. It should be appreciated that although only pressure data for a single patient is shown, the process of detecting decoupling based on pressure signals in the received patient data may be performed for a plurality of patients having had the same or different non-cardiac procedures.

Returning to process 400, after the patient data is analyzed to detect decoupling in act 420, process 400 may proceed to act 430, where a decoupling prediction algorithm or model may be updated based, at least in part, on the decoupling detection and one or more patient characteristics included in the patient data received in act 410. In some embodiments, the decoupling prediction algorithm/model may be updated to associate (e.g., by setting particular weights) various patient characteristics (e.g., age, sex, weight, cardiac history, baseline ejection fraction, required medications, medical history, pressure values measured from previous medical procedures, electrocardiogram values, etc.) to provide as output, a decoupling prediction. The decoupling prediction may take any suitable form. For instance, in some embodiments, the decoupling prediction may include a number (e.g., a percentage) associated with a predicted amount of decoupling for a patient having certain characteristics and/or for patients having had different types of non-cardiac medical procedures. In some embodiments, the decoupling prediction may include a recommendation for the use of an intracardiac blood pump for a non-cardiac medical procedure based on the predicted amount of decoupling (e.g., when the predicted amount of decoupling is above a particular threshold). In other embodiments, the decoupling prediction may include a number (e.g., a percentage) associated with a predicted chance of decoupling during a non-cardiac medical procedure.

When trained on a large amount of patient data, the algorithm/model may learn (e.g., by changing weights in the algorithm/model) patient characteristics that predict the amount of decoupling during a non-cardiac procedure. It should be appreciated that when implemented as a machine learning model, the decoupling prediction model may be implemented as any suitable type of model, examples, of which include a neural-network (e.g., deep learning) based model, a random forest classifier model, a decision trees model (e.g., a gradient boosting decision trees model), or a logistic regression model. Process 400 may then proceed to act 440, where the updated model is output for use in predicting an amount of decoupling for patients not included in the patient data on which the algorithm/model was updated.

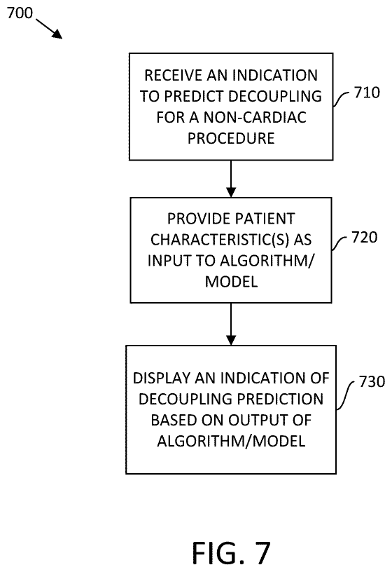

FIG. 7 is a flowchart of a process 700 for predicting an amount of decoupling for a patient scheduled for a non-cardiac procedure (e.g., non-cardiac surgery). Process 700 begins in act 710, where an indication to predict an amount of decoupling for a non-cardiac procedure is received. Process 700 may then proceed to act 720, where one or more patient characteristics (e.g., age, sex, weight, cardiac history, baseline ejection fraction, required medications, medical history, pressure values measured from previous medical procedures, electrocardiogram values, etc.) for a patient are provided as input to an algorithm or model (e.g., a machine learning model) trained to output a decoupling prediction for the patient. In some embodiments, the one or more patient characteristics may be provided from an electronic medical record associated with the patient as input to the algorithm/model. In some embodiments, the decoupling prediction may indicate an amount of decoupling expected to occur during a non-cardiac medical procedure. Process 700 may then proceed to act 730, where an indication of the decoupling prediction output by the algorithm/model may be displayed to a user, such as a physician. In some embodiments, the decoupling prediction may include a recommendation on whether use of an intracardiac blood pump would be beneficial to the patient in association with the non-cardiac medical procedure. In some embodiments, the recommendation may include an indication of a time period during which the intracardiac blood pump should be used (e.g., before, during, and/or after the non-cardiac medical procedure).

It should be appreciated that a performing a decoupling prediction for a patient using one or more of the techniques described herein may be performed at any suitable time. For instance, in some embodiments, the decoupling prediction may be performed when the non-cardiac procedure is scheduled. Such information may enable a physician to determine whether an intracardiac blood pump should be used during the non-cardiac procedure to reduce the risk that the patient will have cardiac complications during the procedure. For instance, the predicted amount of decoupling may be used to assess the likelihood of survival for the patient with or without use of the intracardiac blood pump during the non-cardiac medical procedure.

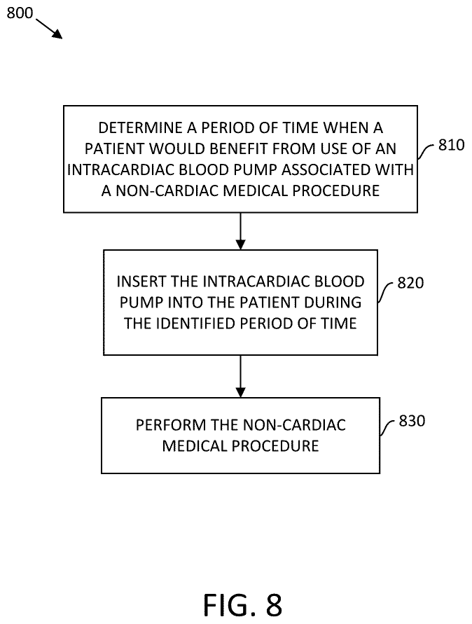

FIG. 8 is a flowchart of a process 800 for treating a patient using an intracardiac blood pump in association with a non-cardiac medical procedure. In that regard, process 800 may be performed if, according to process 700, it has been determined that a patient would benefit from the use of an intracardiac blood pump during the non-cardiac medical procedure (e.g., because the decoupling prediction indicates an amount of expected decoupling above a particular threshold).

In act 810, a determination may be made regarding a period of time during which the patient would benefit from receiving support from the intracardiac blood pump. The period of time may be one or more of before, during, and after the non-cardiac medical procedure. In that regard, an intracardiac blood pump may be used in a variety of ways to lower and/or eliminate risks of the patient experiencing a cardiac event during or after the medical procedure. For example, an intracardiac blood pump may be used before the medical procedure to allow the heart to rest prior to the medical procedure, thus potentially lowering the risk that the heart will subsequently be overcome by the trauma of the medical procedure. Likewise, an intracardiac blood pump may be used during the medical procedure to lower the load on the heart and maintain blood flow through the body, thus potentially lowering the risk of ventricular fibrillation, exacerbated ischemia, myocardial ischemia, pulmonary edema, hemodynamic collapse, cardiac arrest, death, etc., which may occur during the medical procedure. Further, an intracardiac blood pump may be used after the medical procedure to allow the heart to recover, and thus lessen the risk of post-operative cardiac events such as heart attack, ventricular fibrillation, exacerbated ischemia, myocardial ischemia, pulmonary edema, hemodynamic collapse, cardiac arrest, death, etc. Depending on the situation, the intracardiac blood pump may thus be used: (a) only before the procedure; (b) before and during the procedure; (c) before, during, and after the procedure; (d) only before and after the procedure, but not during the procedure; (e) only during the procedure; (f) only during and after the procedure; or (g) only after the procedure.

In act 820, the intracardiac blood pump may be inserted into the patient to provide cardiac support for the period of time determined in act 810. Any suitable way of inserting, positioning, and providing cardiac support using the intracardiac blood pump may be used in this regard, including the methods of providing support that is described herein.

In act 830, the medical procedure may be performed. In some aspects of the technology, acts 820 and 830 may take place simultaneously or their order may be reversed from what is shown in exemplary process 800. For example, in cases where it is determined in act 810 that the intracardiac blood pump is only to be used after the medical procedure, the medical procedure (act 830) may take place before insertion of the intracardiac blood pump (act 820). Likewise, in cases where the intracardiac blood pump is to be used during the medical procedure, the intracardiac blood pump may nevertheless be inserted into the patient (act 820) at some point after the medical procedure (act 830) has begun. In some aspects of the technology, the act of performing the medical procedure may include or commence with placing the patient under anesthesia.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification, unless clearly indicated to the contrary, should be understood to mean “at least one.”

The phrase “and/or,” as used herein in the specification should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

1. A computer-implemented method, comprising:
receiving one or more patient characteristics associated with a patient scheduled for a non-cardiac medical procedure;
providing the one or more patient characteristics as input to a machine learning model trained to output a decoupling prediction;
processing, using at least one computer processor, the one or more patient characteristics using the machine learning model to output a decoupling prediction for the patient, wherein the decoupling prediction is associated with the non-cardiac medical procedure; and
displaying, on a user interface, an indication of the decoupling prediction for the patient output from the machine learning model.

2. The computer-implemented method of claim 1, wherein the one or more patient characteristics include one or more of physical characteristics, medical history information, medication information, or physiological metrics associated with the patient.

3. The computer-implemented method of claim 1, wherein receiving one or more patient characteristics comprises receiving the one or more patient characteristics from an electronic medical record associated with the patient.

4. The computer-implemented method of claim 1, wherein the machine learning model is trained using training data from a plurality of patients having undergone different types of non-cardiac medical procedures.

5. The computer-implemented method of claim 1, wherein displaying an indication of the decoupling prediction for the patient comprises displaying a predicted amount of decoupling that the patient is likely to experience during the non-cardiac medical procedure.

6. The computer-implemented method of claim 5, wherein the non-cardiac medical procedure has an expected procedure duration, and wherein displaying a predicted amount of decoupling comprises displaying the predicted amount of decoupling as a percentage of the expected procedure duration.

7. The computer-implemented method of claim 5, further comprising:
determining, when the predicted amount of decoupling exceeds a threshold value, that the patient would benefit from use of an intracardiac blood pump in association with the non-cardiac medical procedure; and
displaying, on the user interface, a recommendation associated with the use of the intracardiac blood pump.

8. The computer-implemented method of claim 7, wherein the recommendation includes a time period during which use of the intracardiac blood pump would be beneficial to the patient.

9. The computer-implemented method of claim 8, wherein the time period includes one or more of before the non-cardiac medical procedure, during the non-cardiac medical procedure, or after the non-cardiac medical procedure.

10. (canceled)

11. A method of treating a patient, the method comprising:
receiving a decoupling prediction for a patient scheduled for a non-cardiac medical procedure, wherein the decoupling prediction is output from a trained machine learning model in response to providing one or more patient characteristics associated with the patient as input;
determining, based on the decoupling prediction, a period of time when the patient would benefit from use of an intracardiac blood pump associated with the non-cardiac medical procedure;
inserting the intracardiac blood pump into a heart of the patient during the determined period of time; and
performing the non-cardiac medical procedure to treat the patient.

12. (canceled)

13. The method of claim 11, wherein the one or more patient characteristics are provided as input to the trained machine learning model from an electronic medical record associated with the patient.

14. The method of claim 11, wherein the trained machine learning model is trained using training data from a plurality of patients having undergone different types of non-cardiac medical procedures.

15. The method of claim 11, wherein the period of time includes one or more of before the non-cardiac medical procedure, during the non-cardiac medical procedure, or after the non-cardiac medical procedure.

16. The method of claim 11, further comprising:
tracking during the non-cardiac medical procedure an amount of decoupling experienced by the patient; and
retraining the trained machine learning model based, at least in part, on the amount of decoupling experienced by the patient and the one or more patient characteristics.

17. A computer-implemented method for training a machine learning model to predict decoupling during a non-cardiac medical procedure, the computer-implemented method comprising:
receiving patient data for a plurality of patients, wherein each of the plurality of patients underwent a non-cardiac medical procedure associated an intracardiac blood pump;
analyzing, by at least one hardware processor, the patient data to detect one or more decoupling events during the non-cardiac medical procedure associated with each patient of the plurality of patients;
training the machine learning model based, at least in part, on the one or more decoupling events detected during a non-cardiac medical procedure and one or more patient characteristics associated with the patient associated with the non-cardiac medical procedure; and
outputting the trained machine learning model for predicting decoupling in one or more patients not associated with the patient data.

18. The computer-implemented method of claim 17, wherein the patient data includes the one or more patient characteristics associated with a patient and pressure information sensed by and/or derived from one or more pressure sensors of a mechanical circulatory support device that includes the intracardiac blood pump.

19. The computer-implemented method of claim 18, wherein the pressure information includes a left ventricular pressure signal and an aortic pressure signal.

20. The computer-implemented method of claim 19, wherein analyzing the patient data to detect one or more decoupling events comprises detecting a decoupling event when a peak of the aortic pressure signal is greater than a peak of the left ventricular pressure signal within a cardiac cycle.

21. The computer-implemented method of claim 20, further comprising:
determining an amount of decoupling for the patient during the non-cardiac medical procedure based on the detected one or more decoupling events,
wherein training the machine learning model based, at least in part, on the one or more decoupling events comprises training the machine learning model based, at least in part, on the amount of decoupling for the patient during the non-cardiac medical procedure.

22. The computer-implemented method of claim 17, wherein receiving patient data for a plurality of patients comprises receiving the patient data from corresponding electronic medical records associated with the plurality of patients.
